(19) United States
(12) Patent Application Publication
(10) Pub. No.: US 2006/0106282 A1
(43) Pub. Date: May 18, 2006

(54) Pulsed infrared imaging and medical intervention system

(75) Inventor: John L. Bala, Pomfret Center, CT (US)

Correspondence Address: ALIX YALE & RISTAS LLP, 750 MAIN STREET, SUITE 1400, HARTFORD, CT, 06103, US

(73) Assignee: Micro Invasive Technology, Inc.

(21) Appl. No.: 10/987,063

(22) Filed: Nov. 12, 2004

(51) Int. Cl. A61B 1/06

(52) U.S. Cl. 600/108; 600/181; 600/178

(57) ABSTRACT

The present invention in a preferred form uses pulse technology to produce wavelengths of light within the infrared spectrum in order to selectively view surgical sites normally occluded by conditions such as smoke, fluids, tissues, and/or haze and to direct laser energy to the surgical site. The invention allows selected wavelengths of infrared light to be directed for the purpose of illumination and imaging of a surgical site. The invention also allows laser energy to be directed to the imaged site. The imaged site can be displayed remotely on, for instance, a viewing screen.

### FIELD OF THE INVENTION

This invention generally relates to medical bioimaging and intervention technology, and in particular to medical bioimaging technology utilizing infrared imaging light and laser radiation intervention.

### BACKGROUND OF THE INVENTION

The ability to view interior portions of a patient's body during a surgical medical procedure is invaluable for efficacious surgical intervention. Conventionally, devices for viewing the interior of a patient's body during a surgical procedure utilize fiberoptic light guides. These conventional light guides allow areas within the body cavity to be both illuminated and visualized through an eyepiece. These conventional systems utilize continuous (CW) light sources that are coupled to an illumination conduit by a light guide and an optical connector located at or near the top of the illumination device. The designers of light sources for use in conventional systems are typically concerned with only light in the visible wavelengths.

For many years, devices such as endoscopes have been used to provide an observer with images from within the human body through an eyepiece. In more recent times, digital cameras with their increased resolution and image quality have replaced direct viewing with the eye. The digital camera allows for enhanced real time viewing of the surgical site, and allows for such things as digital image capture for later analysis by a physician or surgical team. As the sophistication and quality of the digital camera systems have been increased, so has the ability to image internal features of the human body with greater precision and accuracy. This precision and accuracy is required for observation of highly compact and complex surgical sites.

Lasers, for surgical intervention have also played an increasingly useful role throughout the medical community. In the past decade, the medical community has increasingly turned to the versatile laser for use in medical procedures. However, since lasers often create byproducts such as haze or smoke due to their high energy outputs, full implementation of laser technology has been unachievable. For example, smoke is often generated when tissue at a surgical site is obliterated by laser energy. This smoke or haze may prevent additional applications of laser energy to the surgical site by visually occluding the site. In addition, the surgical site may become flooded, coated, covered, or otherwise associated with fluids and/or tissues. This association with fluid and/or tissue can prevent the precise application of laser energy and may prevent a surgical site from being readily available to the surgeon. For example, blood may coat or cover an area to the extent it will prevent precise visual identification of the structures and landmarks needed to reference the intervention location.

Currently there is no single system in place for simultaneously imaging at light ranges outside the visible wavelengths and for directing laser intervention energy.

### SUMMARY OF THE INVENTION

Briefly stated, the present invention in a preferred form is generally directed toward a device using pulse technology to produce wavelengths of light within the infrared spectrum in order to view surgical sites normally occluded by conditions such as smoke, fluids, tissues, and/or haze and to direct laser energy to the viewed surgical site. The invention also allows selected wavelengths of infrared light to be directed for the purpose of illumination and imaging of a surgical site. The invention additionally allows laser energy to be accurately directed to the imaged site. In use, light can enter a body of an endoscope or similar device through a light channel such that a specific wavelength can be selected which is different from conventional illumination wavelengths. The selection of a specific range of infrared wavelengths can be accomplished by use of filters and/or gratings located on a light source such as a flashtube or on such devices as a filter wheel. Alternatively, a continuously variable filter wheel may be used in order to select the desired wavelength of light.

An object of the invention is to allow the viewing of and direction of laser energy toward structures located behind occluding materials such as haze, smoke, tissues and/or blood.

Another object of the invention is to allow users to image and to direct laser energy to imaged structures occluded by tissues such as veins and/or artery walls.

A further object of the invention is to provide imaging and laser intervention technology, which is cost effective for use in medical procedures.

### BRIEF DESCRIPTION OF THE DRAWINGS

The present invention may be better understood and its numerous objects and advantages will become apparent to those skilled in the art by reference to the accompanying drawings in which:

FIG. 1 is a simplified partial perspective view of an imaging and intervention system in accordance with the present invention.

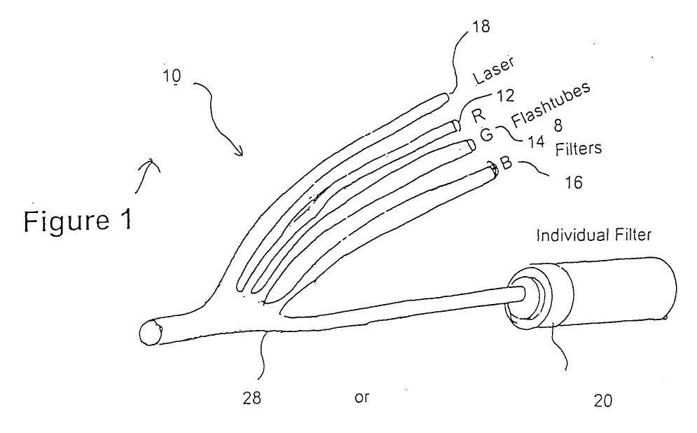

FIG. 2 is a simplified perspective view of devices used to vary the wavelength of light in accordance with the present invention.

FIG. 3 is a graph showing various wavelengths expressed in nanometers of light output.

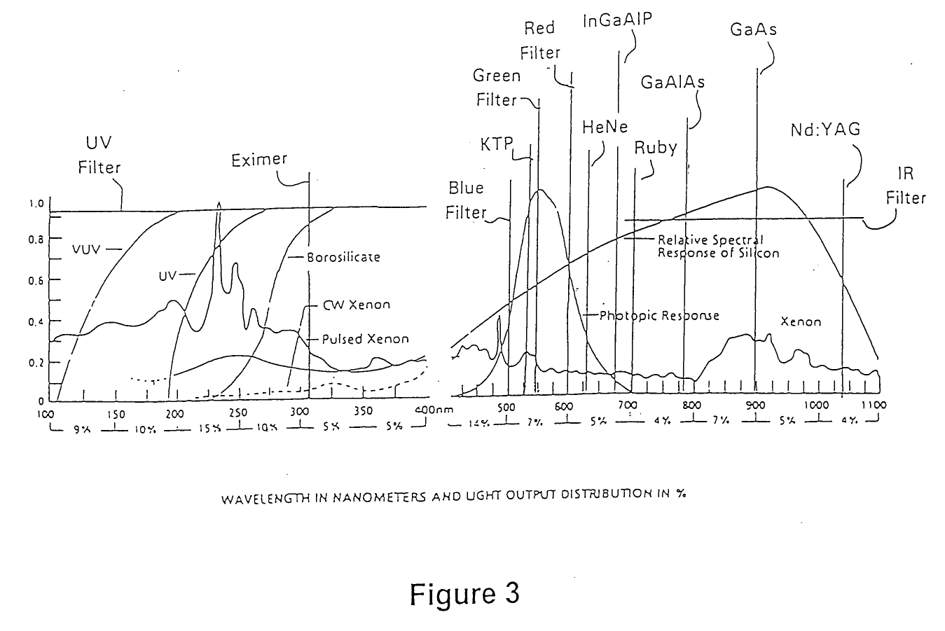

FIG. 4 is a simplified perspective view of an endoscope imaging and intervention system in accordance with the present invention.

### DETAILED DESCRIPTION OF THE PREFERRED EMBODIMENT

With reference to the drawings wherein like numerals represent like parts throughout the several figures, an imaging and intervention system in accordance with the invention is designated by the numeral 10. The device can be used for medical bioimaging within, for example, a patient's abdominal cavity to enhance the visualization of areas of interest. Once the enhanced visualization is achieved, laser energy can be selectively directed to the desired intervention site. For example, a site of surgical intervention may be located within a patient's abdominal cavity such that it cannot be conventionally accessed visually due to intervening vein and/or artery walls. In other cases, the site of surgical intervention may contain malignant tissue which is intertwined with or shares a complex border with sensitive nerve or organ tissue. Initial applications of laser energy can be precisely and accurately directed to such surgical sites, however, interference free imaging is critical for accurate direction of additional applications of the energy.

Biological tissue, fluids, and such things as smoke and haze cause visual distortion and opacity due to the light scattering properties of these materials. The use of particular wavelengths of light can be beneficial for imaging through turbid or opaque materials since different materials will typically have different light scattering and reflective properties depending on the wavelength of light used. These differing absorptive and reflective properties of fluids and/or tissues often mean that such materials be made essentially transparent at certain selected wavelengths which are often unique to that fluid or tissue. In addition, tissues can often be identified based on the differing optical properties they possess when illuminated at differing wavelengths of light.

In one embodiment of the invention, infrared wavelengths of illumination light pass through tissues and/or fluids to the surgical site. The illuminated surgical site can be viewed and imaged with, for instance, a CCD camera which can detect reflected infrared light. Intervening tissue, fluid, smoke and/or other materials having been made effectively transparent through selection of certain wavelengths of infrared illumination light do not interfere with viewing and imaging of the surgical site. These certain wavelengths of infrared light can be easily found either through trial and error during the procedure, or through reference to well known reflective and absorptive properties. Intervention energy can then be directed to precise and accurate locations within the site.

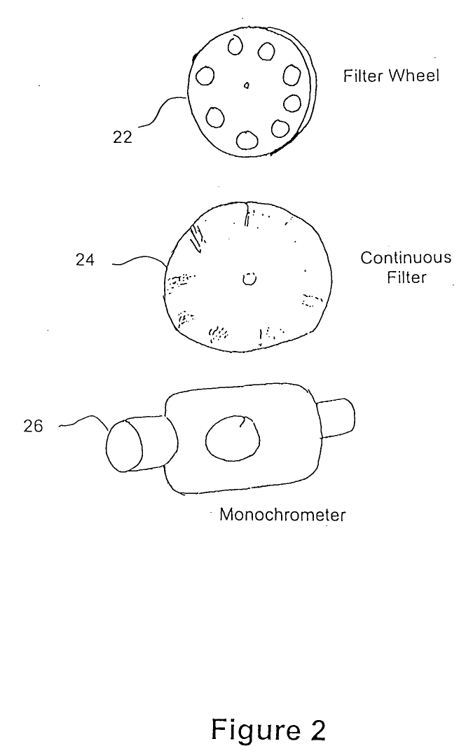

In one embodiment of the invention, the imaging and intervention system 10 may be an endoscope having inputs for light sources such as flashtubes. The flashtubes can include a red flashtube 12, a green flashtube 14 and a blue flashtube 16 for generating visible wavelengths of light. An intervention laser input 18 is also present. There can also be a flashtube having a wavelength selection device 20 such as an individual filter 21. The filter 21 may be replaced, in some cases, with an individual filter wheel 22, a continuous filter 24, or a monochrometer 26 as illustratively shown in FIG. 2. The wavelength selection device 20 be linked, for example, to a quintfurcated optical guide unit 28. The quintfurcated fiberoptic channel is connected with a fiberoptic bundle 30 via a connector 27. Energy from the flashtubes 12, 14, 16, the laser 18, and the wavelength selectable device 20 can be selectively directed down pathways contained within the fiberoptic bundle 30. This energy can then be directed out through a probe 32 and can be used to selectively illuminate and surgically intervene with interior portions of a patient's body. A camera 34 is connected to the optical guide 28 by a connector 36. The camera 34 includes, for example, a CCD sensor array. The camera 34 can be selected based on the sensitivity and the resolution required for the range of wavelengths of light to be used.

The probe 32 used may be flexible or rigid, and may have, for example, a diameter of 0.5-10 mm. The selection of the probe 32 may be based on factors related to the distance needed to be traversed within the patient's body, and/or handling characteristics of the probe. The probe 32 is configured such that it allows for output of illuminating light, output of high energy such as a laser emission, and an optical input for receiving images that can be transmitted along a pathway to the camera 34.

For imaging, the flashtubes 12, 14,16, and the wave selective device 20, may be connected via a service cable assembly 38 to a control module 40. Selective use of these flashtubes allow a user to image an area of interest under both visible and infrared wavelengths depending on the surgical procedure. Each flashtube assembly 12, 14, 16 and the wave selective device 20, may include a pulsed xenon light source emitting a light pulse having the equivalent of 100,000 watts of light power with a duration of approximately 10 microseconds as described in U.S. patent application Ser. No. 10/718,771, filed on Nov. 21, 2003 incorporated fully herein by reference. However, other durations and power levels may be selected. The different flashtubes may have differing filters present, for example, a red, green, blue, and an infrared filter. Each flashtube may be connected to the fiberoptic bundle 30 by an optical connector 62.

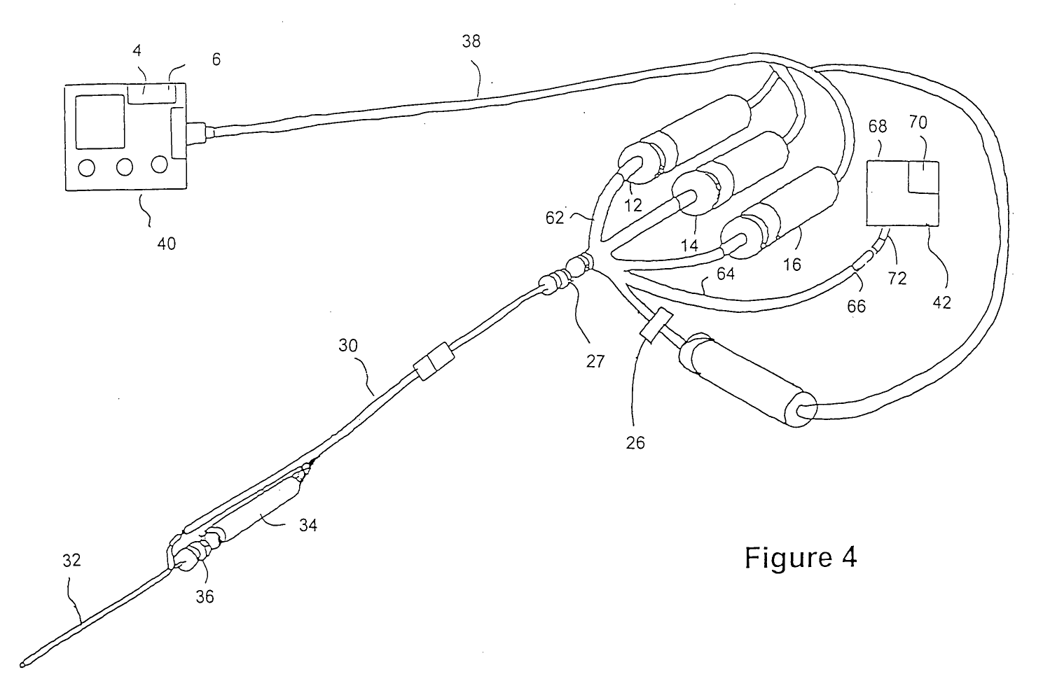

As shown in FIG. 4, the spectral content of the pulsed xenon flashtube is extremely broad. FIG. 4 shows the spectral range about 100 nm to about 1,100 nm. The pulsed xenon flashtube has its highest output in the UV range of 200-300 nm with a peak output at approximately 225 nm. Approximately 35% of the energy is in the ultraviolet spectrum; 26% of the flashtube energy is in the visible spectrum with the highest peaks between 400 and 500 nm. Approximately 16% of the flashtube output is in the infrared region between about 800 and about 1,100 nm. As can be seen from FIG. 4, there are many windows and significant energy present for producing and viewing infrared images based on the xenon technology. Use of light in the infrared wavelength range may be used selectively to illuminate the area of interest. Light of this wavelength range has the ability to pass through and be reflected back through such materials as blood, haze, certain tissues and/or smoke.

A laser generator 42 may be optically connected with the system via a connector 64 so as to allow the transmission of laser radiation through a pathway of the fiberoptic bundle 30.

During the operation of one embodiment of the invention, the light from the flashtube is guided along a pathway in the fiberoptical bundle 30. The light is allowed to pass out through the end of the probe 32. This passed light illuminates areas and a portion of the light may be reflected back from the areas proximate the end of the probe. The reflected near-infrared light is then collected at the probe end and transmitted along an optical pathway to the CCD camera 34. The camera sensor captures the image produced by the reflected light. This image may then be transmitted to a viewing screen, for example, the image may be displayed as a monochrome image. The displayed image may also be shown on a separate monitor or may be used in picture format on the monitor.

In one embodiment of the invention, probe 32 may be about 300 mm long and be constructed of glass or plastic, for example, the probe may be formed mainly from a high refractive glass rod having a diameter of approximately 10 or less millimeters. The rod provides, among other things, an optical pathway, which forms an image tunnel from the distal to the proximal end of the probe 32. The optical pathway also is utilized to direct the path of high energy such as laser emissions. Surrounding the imaging tunnel may be a second optical pathway providing illumination to the surgical sight from a light source. The illumination pathway may be constructed from a clear plastic rod and may form a sleeve on the outside diameter of the imaging tunnel, which acts as, for example, a light pipe. The illumination pathway interfaces with an optical connector such as a fiberoptic annulus, which allows light to be brought into the probe. The light may be generated by a source located in a remote system control unit 40, which is then brought into the probe 32, and then into the body cavity. Aperture stops and carbon black absorbed as coatings may be associated with the image tunnel in order to intercept and attenuate light rays, which enter the tunnel from outside of the field of view. Rays that are in the field of view preferably may enter the tunnel. Rays originating from outside the field of view, when aperture stops and/or carbon black are used, are absorbed or prevented from propagating into the image tunnel by reflection. The absorption prevents veiling glare and a reduction of image contrast. The probe 32 allows for the transfer of the images from the end of the probe 32 to an electronic camera 34 located in the housing of a sensor module. In some cases the aperture stops are cut into the outer diameter of the glass rod by means of diamond turning process.

The fiberoptics of the system may be cabled to the light source located in the system control module 40. The advantage of the pulse xenon source of very short duration is that temperatures are not elevated above temperature ranges that are safe for surrounding tissue. High non-pulsed outputs, while being useful for illumination, also result in a harmful elevation in temperature. Short duration pulsed energy is difficult for the human eye to respond to. The CCD camera while also allowing for the monitoring of spectra outside the human limit of detection also allows the pulses of intense light to be utilized. In one embodiment of the invention, the system displays and freezes the image between each pulse with the most recent image displayed on, for example, a video monitor. The display can be updated, for example, 30 times per second providing the equivalent of a current integrated image. The pulse repetition rate can be determined experimentally for optimal image quality and could range between about 30 to about 60 pulses per second.

The pulsed xenon flashtube may be connected optically and electronically through an integrated cable between the system control module 40 and the sensor module 34. Twist lock connectors may be located at each module for ease of cable replacement. These cables typically may receive the greatest use and, therefore, may be required to be replaced.

Digital control is available in one embodiment of the invention to vary the intensity of the illumination source via manual adjustment and the rate at which the light pulses are generated. CCD sensors can be chosen that are sensitive to infrared in, for example, the 700 to 1200 nanometer range. Various filters can be installed along the optical pathway running between the light source and the sensor itself such that only infrared light is used for the illumination of the sight to be imaged or a filter may be put in place which only allows reflected in the infrared range to reach the sensor.

While the preferred embodiments of the foregoing invention have been set forth for the purposes of illustration, the foregoing description should not be deemed a limitation of the invention herein. Accordingly, various modifications, adaptations and alternatives may occur to one skilled in the art without departing from the spirit and scope of the present invention.

1. An imaging and intervention endoscope system comprising:
a high energy intervention unit having a high energy output;
an illumination unit having an illumination output and a wavelength selector, said wavelength selector having an illumination input and an infrared illumination output;
an image sensor having an image input and an image output;
an optical probe having an optical connector and an end;
an optical bundle connected to the optical probe including a high energy pathway for receiving and conducting the high energy output from the high energy intervention unit to the probe end, an illumination pathway for receiving and conducting the illumination output from the illumination unit to the wavelength selector illumination input and receiving and conducting infrared illumination from the infrared illumination output to the probe end, an image pathway for receiving and conducting an image from the probe end to the image sensor; and
an image display unit having a screen that displays images from the image sensor image output.

2. The imaging and intervention endoscope system of claim 1 wherein the high energy illumination unit is a pulse xenon flashtube having an optical spectrum from about 190 nm to greater than 1200 nm.

3. The imaging and intervention endoscope system of claim 1 wherein the wavelength selector is a filter that allows the passage of selected light wavelengths in the range of about 700 nm to about 1200 nm.

4. The imaging and intervention endoscope system of claim 1 wherein the wavelength selector is a filter wheel with a plurality of filters that each allow the passage of a range of light wavelengths which wavelengths are selected from a range of about 700 nm to about 1200 nm.

5. The imaging and intervention endoscope system of claim 2 wherein the high energy intervention unit is a laser.

6. The imaging and intervention endoscope system of claim 1 wherein the wavelength selector is a continuous filter wheel having portions which allow passage of light wavelengths in a range of about 700 nm to about 1200 nm.

7. The imaging and intervention endoscopic system of claim 1 wherein the wavelength selector is a grating that allows the passage of selected light wavelengths in the range of about 700 nm to about 1200 nm.

8. The imaging and intervention endoscope system of claim 1 wherein the illumination output is a pulsed output.

9. The imaging and intervention endoscope system of claim 1 wherein the illumination pathway surrounds the imaging pathway, and said imaging pathway has a portion that is coextensive with the high energy pathway.

10. An imaging and intervention endoscopic system comprising:
a multi-path optical array having guide portions for a high energy output, an illumination output, and an image input;
a sensor for receiving the image input and generating an image signal;
a probe optically connected to the multi-path optical array and having an end for receiving the image input and conducting and emitting high energy output and illumination output; and
a wavelength limiter associated with the multi-path optical array such that substantially only selected illumination output in the infrared wavelengths may pass through the limiter.

11. The imaging and intervention endoscope system of claim 10 wherein the probe has a portion which includes a high refractive glass rod having a diameter of about 1 mm.

12. The imaging and intervention endoscope system of claim 10 wherein the sensor is a CCD sensor.

13. The imaging and intervention endoscope system of claim 10 wherein the wavelength limiter is a filter that allows the passage of selected light wavelengths in the range of about 700 nm to about 1200 nm.

14. The imaging and intervention endoscope system of claim 10 wherein the wavelength limiter is a filter wheel with a plurality of filters that each allow passage of a range of light wavelengths which wavelengths are in a range of about 700 nm to about 1200 nm.

15. The imaging and intervention endoscope system of claim 10 wherein the wavelength limiter is a continuous filter wheel having portions which allow passage of light wavelengths in a range of about 700 nm to about 1200 nm.

16. The imaging and intervention endoscope system of claim 10 wherein the wavelength limiter is a grating that allows selected light wavelengths to pass in the range of about 700 nm to about 1200 nm.

17. The imaging and intervention endoscope system of claim 10 wherein the illumination output is a pulsed output.

18. An imaging and intervention endoscope system comprising:
an optical assembly having a plurality of optical pathways that allow a laser emission to be directed to a remote site, an infrared illumination emission to be directed to the remote site, and a reflected infrared emission to be collected from the remote site and delivered to a digital image sensor; and
a detachable probe unit that is connectable with the plurality of optical pathways, said probe having an end through which the laser emission, infrared emission, and reflected infrared emission pass.

19. The imaging and intervention endoscope system of claim 18 wherein the near-infrared emission has a wavelength in the range of about 800 nm and about 1100 nm.

20. The imaging and intervention endoscope system of claim 18 wherein the digital image sensor is a CCD sensor.
